(19) United States
(12) Patent Application Publication
(10) Pub. No.: US 2009/0104284 A1
(43) Pub. Date: Apr. 23, 2009

(54) Topically Applied Chitosan Composition for Pain Relief and Boosting Immune System

(76) Inventors: Herbert Harrell Gunn, Jr., Atlanta, GA (US); Connie G. Gunn, Atlanta, GA (US)

Correspondence Address: BERENATO, WHITE & STAVISH, LLC, 6550 ROCK SPRING DRIVE, SUITE 240, BETHESDA, MD, 20817, US

(21) Appl. No.: 12/249,663

(22) Filed: Oct. 10, 2008

Related U.S. Application Data
(60) Provisional application No. 60/981,401, filed on Oct. 19, 2007.

(51) Int. Cl. A61K 33/30

(52) U.S. Cl. 424/642

(57) ABSTRACT

A composition of matter that when applied topically relieves pain in the area where it is applied (i.e. knees, back, elbows, heels, etc.). The composition contains chitosan, canola oil, sea salt minerals (chloride ion, sulfate ion, calcium, potassium, zinc, sodium, magnesium and nickel), and water. To make the composition, the ingredients should be at room temperature. Dissolve 7-8 grams of medical grade or edible grade chitosan in 1 liter of canola oil. Dissolve 2 tsp of sea salt containing the following minerals (chloride ion, sulfate ion, calcium, potassium, zinc, sodium, magnesium and nickel) in 50ml of distilled water. Pour the sea salt solution into the chitosan-canola oil mixture. The chitosan-canola oil is hydrophilic and absorbs the sea salt water into the composition negating the water-oil interface. Shake well for 10 minutes or mechanically blend for 5 minutes. Apply generously to skin over affected area. The same ratios apply to larger batches. The chitosan-oil-water-mineral composition permits the ingredients to cross tissue barriers to effect skin, connective tissue, tendon, ligaments, cartilage and bone to relieve pain, stop inflammation and heal inflamed underlying tissue. The respective components of the chitosan-oil-water-mineral composition will not cross the pericutaneous tissue barriers alone.

### BACKGROUND OF THE INVENTION

1. Field of the Invention

The invention relates generally to a topically applied chitosan composition for pain relief and for boosting the immune system.

2. Background

Currently there have been a number of attempts to use chitosan for various purposes, such as a plant growth enhancer, as an additive in the water filtration process, field bandages, in tablet form as a fat attractor, as a matrix material in bone grafts.

There have been attempts to use certain compositions containing chitosan including: (U.S. Patent Application Pub. No.: US2007/0128258) as an oil in water emulsion gel; (U.S. Pat. No. 7,166,281, B2) as an abrasive ingredient and an anti-itch enzyme, dispersed in a carrier containing effective amounts of emulsifiers and/or dispersants and may take the form of powder, liquid, or paste and may also contain an anesthetic and/or antibiotic; as a cosmetic dermatological or pharmaceutical preparation; (U.S. Patent Application Pub. No.: US 2006/0147390); for treatment of infected skin and mucus membrane (U.S. Patent Application Pub. No.: U.S. 2006/0105000 A1.); for treatment of chronic inflammatory disease (U.S. Patent Application Pub. No.: U.S. 2005/0090553 A1); for treatment of a skin disorder (U.S. Pat. No. 5,874,074.). U.S. Pat. No. 5,912,000 describes chitosan induced immunopotentiation.

Canola oil has been used mainly in cooking oil. Likewise, sea salt has a variety of uses, including cooking and myriad treatments and compositions.

It would be desirable to have a composition that is inexpensive, odorless and hypoallergenic that can be applied topically directly over painful areas/regions of the body that will reduce pain, retard inflammation, impede bacterial growth and boost the immune system.

Furthermore, it would be desirable to have a composition that is made from chitosan, which is hypoallergenic, has natural anti-bacterial and anti-inflammatory properties and is one of the strongest immune system boosters known.

Still further it would be desirable to have a composition in which some of the chitosan dries into a matrix pattern that can assist scar tissue formation. This leads to improved scar formation by creating faster more organized scars, lessening of infection, retarding inflammation and more effective fighting of bacterial infection.

Still further it would be desirable to have chitosan transported into all the tissues underlying the skin including the bone. When in the body, chitosan(N-acetyl-D-glucosamine)biodegrades into glucosamine. Glucosamine is transported under the skin and can affect local tissues directly.

Glucosamine is currently being used orally to treat and relieve osteoarthritis and assist in cartilage formation. Transportation of glucosamine precursors directly to the site of use after topical application is a more effective way of getting the glucosamine to where it is needed and is currently not being used except in this invention.

Still further it would be desirable to be able to transport minerals under the skin. These minerals are hypoallergenic and can assist painful conditions in a beneficial way by slowing the transmission of painful nerve impulses.

Further, minerals can decrease transmission of muscle depolarization and repolarization impulses, which decreases muscle spasms, relaxes muscles and stops muscle tightness.

Still further it would be desirable to get water into the tissue spaces to re-hydrate the tissue spaces. Extra water can be used to fight local infection and inflammation and re-hydrate tissues in order to combat aging, combat wrinkling, local infection and drying.

Still further it would be desirable to have canola oil transported into the subcutaneous spaces in order to help strengthen and rebuild the cell walls of tissues.

Still further it would be desirable to have sea salt minerals transported pericutaneously to assist in the rebuilding of tissue, facilitate transmission of muscle and nerve impulses, supply electrolytes and help enzymatic reactions.

Still further it would be desirable to have a composition in which minute amounts of the composition when applied to the spinal column over a period of time can cause hydration of the intervertebral disc and decrease disc space narrowing and tissue shrinkage of old age and illness.

Still further it would be desirable to have all the ingredients cross into the subcutaneous area together because they are all hypoallergenic, they are all in the body already and can replace elements that are being used for other purposes.

Therefore, there exists a need in industry for a chitosan-based pain reliever with mineral ions, water and canola oil that also has effect as an inflammation retardant, immune system booster and antibacterial composition. There also exists a need for a chitosan-based product which is safe to use, inexpensive to make and improves lesions in all tissue layers of the body down to the bone.

Still further there exists a need for a composition that, when the ingredients are blended as previously described, potentiate each other and become more powerful than they would be if each ingredient were used alone.

### SUMMARY OF THE INVENTION

The present invention advantageously fills the aforementioned deficiencies by providing a topically applied chitosan composition for pain relief and boosting immune system response.

The present invention is an all natural odorless, topical chitosan composition for pain relief which is made of canola oil (93 to 94% by weight), chitosan (0.65 to 0.85% by weight), water (4.7 to 5.1% by weight), chloride ion (0.34 to 0.54% by weight), sulfate ion (0.009 to 0.0201% by weight), calcium ion (0.0009 to 0.00002% by weight), potassium ion (0.0012 to 0.0032% by weight), zinc ion (0.0000009 to 0.0032% by weight), sodium ion (0.005 to 0.007% by weight), magnesium ion (0.008 to 0.01% by weight), nickel ion (0.000001 to 0.00002% by weight).

The following method can be used to make the composition at room temperature (78 F.): add 7-8 grams of medical grade or edible grade chitosan in 1 liter of canola oil. Dissolve 2 tsp of sea salt containing the following minerals (chloride ion, sulfate ion, calcium, potassium, zinc, sodium, magnesium and nickel) in 50 ml of distilled water. Pour sea salt solution into the chitosan-canola oil composition. The chitosan-canola oil is hydrophilic and absorbs the sea salt solution into the composition negating the water-oil interface. Shake well for 10 minutes. Apply generously to skin over affected/afflicted areas of body. The same ratios apply to larger batches.

Chitosan, canola oil, water and mineral ions must all be used in the described way in order to cross into all the tissue spaces together and affect all of the tissues down to the bone.

It should be noted that by lowering the concentration of minerals, the invention can be used on open wounds for homostasis and as a matrix for scar formation.

The invention can be used as a solid with the consistency of butter (canola butter) or an oil (canola oil).

Fragrances may be added as long as these fragrances are hypoallergenic and will not interfere with the ability of the composition to cross over into the subcutaneous areas.

Varying the ion concentration more toward neutral pH by decreasing the amount of ions used can make the invention more soothing on open wounds.

Varying the amount of the water in conditions causing a decrease in the water concentration can hydrate the area more and can be helpful in healing the treated condition.

The present invention is unique when compared to other known chitosan compositions because when used in the aforementioned composition of chitosan-canola oil-water-sea salts the invention is odorless, inexpensive, easy to assemble at room temperature, produces the surprising ultimate results of pain relief, muscle spasms relief, anti-inflammatory, antibacterial and boosts the immune system, making joints more mobile and feeling more lubricated when used topically.

None of the ingredients has a negative effect on the body if used in the composition as described, as all the ingredients are hypoallergenic and natural.

All the ingredients when used together cross all the tissue barriers together down to the bone where they also affect bony architecture.

Finally, it is an object of the present invention to provide a topically applied chitosan composition for pain relief and boosting immune system that does not suffer from any of the problems or deficiencies associated with prior solutions.

The present invention now will be described more fully hereinafter with reference to the accompanying drawings, which are intended to be read in conjunction with both this summary, the detailed description and any preferred and/or particular embodiments specifically discussed or otherwise disclosed. This invention may, however, be embodied in many different forms and should not be construed as limited to the embodiments set forth herein; rather, these embodiments are provided by way of illustration only and so that this disclosure will be thorough, complete and will fully convey the full scope of the invention to those skilled in the art.

### DETAILED DESCRIPTION OF THE INVENTION

In its most complete version, the composition according to the present invention is made using the following steps: have the ingredients at approximately room temperature (78 F.). Dissolve 7-8 grams of medical grade or edible grade chitosan in 1 liter of canola oil. Dissolve 2 tsp of sea salt containing the following minerals (chloride ion, sulfate ion, calcium, potassium, zinc, sodium, magnesium and nickel) in 50 ml of distilled water. Pour sea salt solution into the chitosan-canola oil composition. The chitosan-canola oil is hydrophilic and absorbs the sea salt solution into the composition negating the water-oil interface. Shake well for 10 minutes. Apply generously to skin over affected area/regions of the body. The same ratios apply to larger batches.

The present invention is an all natural odorless, topical chitosan composition for pain relief which is made of canola oil (93 to 94% by weight), chitosan (0.65 to 0.85% by weight), water (4.7 to 5.1% by weight), chloride ion (0.34 to 0.54% by weight), sulfate ion (0.009 to 0.0201% by weight), calcium ion (0.0009 to 0.00002% by weight), potassium ion (0.0012 to 0.0032% by weight), zinc ion (0.0000009 to 0.0032% by weight), sodium ion (0.005 to 0.007% by weight), magnesium ion (0.008 to 0.01% by weight), nickel ion (0.000001 to 0.00002%).

It should be noted that these components, chitosan-oil-water-minerals, when used together permit all the ingredients to cross all the tissue barriers to effect skin, connective tissues, tendons, ligaments, etc. Together they retard and stop infection and inflammation in the tissues they encounter. They boost the immune system, which is the system that heals disease processes and fights infection and inflammation.

Chitosan is the most powerful active ingredient in the present composition. Chitosan is a linear polysaccharide composed of randomly distributed-(1-4)-linked D-Glucosamine (deacetylated unit) and N-acetyl-D-Glucosamine (acetylated unit). It has a number of known commercial and possible biomedical uses.

Chitosan is the second most abundant biopolymer in nature, the first being cellulose which is found in plants. It is a glucose-based unbranched polysaccharide mostly seen in the exoskeletons of crustaceans and insects as well as the cell walls of some bacteria and fungi. Chitosan is to animal cells as cellulose is to plants cells. It exerts its influence on all the cells and tissues but mainly on the cell wall and immune system.

In the body, the immune system is the chief system for fighting disease processes. The immune system makes immunoglobulins (antibodies), i.e. IgM, IgG, IgA, etc. In the body, the immune system is the chief system for fighting disease processes. Chitosan has been found to increase the Immunoglobulin G (IgG). The antibody IgG is the only isotype that can pass through the human placenta, thereby providing protection to the fetus in utero. Along with IgA secreted in the breast milk, residual IgG absorbed through the placenta provides the neonate with humeral immunity before its own immune system develops. It can bind to many kinds of pathogens, for example viruses, bacteria, and fungi, and protects the body against them by agglutination and immobilization, complement activity (classical pathway), oponization for phagocytosis and neutralization of their toxins. It also plays an important role in antibody-dependent cell mediated cytotoxicity (ADCC). IgG is also associated with Type II and Type III Hypersensitivity. Chitosan is rapidly adsorbed in to the tissue spaces with favorable results.

Chitosan is not a protein. Most allergic reactions are caused by allergies to proteins. Chitosan is a polysaccharide (a sugar), which gives it extremely low allergic potential. Further, it biodegrades into glucosamine when in the body.

Chitosan is also a bio-adhesive and readily binds to negatively-charged surfaces such as mucosal membranes. Chitosan enhances the transport of polar drugs across epithelial surfaces, and is biocompatible and biodegradable. As such, purified quantities of chitosans are available for biomedical applications.

Unfortunately chitosan, as it is found in crab, insect and worm shell is not used very much in the Western diet. This invention is a way of supplying much needed chitosan, minerals, canola oil and water to the body without oral ingestion. Because of chitosan's importance in immune system boosting, its ability to help the immune system in making antibodies, its lack in the western diet and its ability to cross into the tissue spaces, its ability to boost IgG and other antibody biosynthesis, it likewise makes this invention important in general health and especially in pregnant women.

The canola oil in this chitosan mixture is the adjuvant (or carrier) to allow the chitosan, water, minerals and canola oil to cross the skin barrier and affect the underlying tissue. Canola oil promotes good health due to its very low saturated fat and high monounsaturated fat content, and beneficial omega-3 fatty acid profile. Monounsaturated fats are most desirable to use in the human body. They have far less undesirable side effects than saturated fats when used in the body. They help in cell wall formation and repair. The Canola Council of Canada states that it is completely safe and is the “healthiest” of all commonly used cooking oils. Researchers have found canola oils antioxidant activity was much greater than that of well-known antioxidants, including alpha-tocopherol, vitamin C, beta-carotene, rutin, and quercetin.

Canola oil has beneficial effects on lowering blood pressure, lowering the risk of cell mutation, which causes cancer. Regular substitution of canola oil for other dietary lipid sources may assist in reducing the likelihood of transient ischemic events leading to life-threatening cardiac arrhythmias. Canola oil has demonstrated its benefits on platelet functions in trials.

It should be noted that the invention can be used as a solid with the consistency of butter (canola butter) or as an oil (canola oil).

The beneficial effects of water are well known. The body is 55% to 85% water. The body uses the circulatory system to get water to some tissues. Even with this high concentration of water, tissues that have a poor blood supply and consequently a poor water supply can become dehydrated. Cartilage and intervertebral disc both have a poor blood supply and consequently suffer from dehydration. This dehydration gets severe as a person ages. It is important to find a way to get water to the cartilage and intervertebral disc. The chitosan-canola oil-mineral-water composition can carry its water directly to where the body needs it without going through the circulation. This presents a unique method of disc and cartilage hydration.

Water is also a common chemical substance that is essential for the survival of all known forms of life. From a biological standpoint, water has many distinct properties that are critical for the proliferation of life that set it apart from other substances. It carries out this role by allowing organic compounds to react in ways that ultimately allow replication. All known forms of life depend on water. Water is vital both as a solvent in which many of the body's solutes dissolve and as an essential part of many metabolic processes within the body. Metabolism is the sum total of anabolism and catabolism. In anabolism, water is removed from molecules (through energy requiring enzymatic chemical reactions) in order to grow larger molecules (e.g. starches, triglycerides and proteins for storage of fuels and information). In catabolism, water is used to break bonds in order to generate smaller molecules (e.g. glucose, fatty acids and amino acids and biodegrading chitosan into glucosamine to be used for fuels for energy use or other purposes). Water is thus essential and central to these metabolic processes. Therefore, without water, these metabolic processes would cease to exist, leaving us to muse about what processes would be in its place, such as gas absorption, dust collection, etc.

Water is also central to photosynthesis and respiration. Photosynthetic cells use the sun's energy to split off water's hydrogen from oxygen. Hydrogen is combined with CO2 (absorbed from air or water) to form glucose and release oxygen. All living cells use such fuels and oxidize the hydrogen and carbon to capture the sun's energy and reform water and CO2 in the process (cellular respiration). Water is also central to acid-base neutrality and enzyme function.

It should be noted that by varying the amount of the water in conditions causing a decrease in the water concentration can hydrate the area more and can be helpful in healing the condition being treated.

Minerals from salt(s) are needed to run the nerve and muscle processes of the body. There are usually enough minerals in the body in healthy conditions; however in disease and stressful conditions replacing depleted minerals becomes extremely important especially in local areas where pain exists or there is inflammation or infection. It should be noted that dietary minerals are the chemical elements required by living organisms, other than the four elements carbon, hydrogen, nitrogen and oxygen, which are present in common organic molecules. The term mineral is archaic, since the intent of the definition is to describe ions, not chemical compounds or actual minerals. Furthermore, once dissolved, so-called minerals do not exist as such, sodium chloride breaks down into sodium ions and chloride ions in aqueous solution.

Here are some of the properties of the mineral ions used in the present invention: Calcium is for muscle and digestive system health, builds bone, neutralizes acidity, clears toxins, and helps the blood stream. Magnesium is required for processing ATP and related reactions (health, builds bone, causes strong peristalsis, increases flexibility, increases alkalinity).

Potassium is a required electrolyte for heart and nerve health. Sodium is an electrolyte. Nickel is present in urease. Sulfur is required for three essential amino acids and many proteins and cofactors (skin, hair, nails, liver, and pancreas health). Zinc is required for several enzymes such as carboxypeptidase, liver alcohol dehydrogenase, carbonic anhydrase. Zinc is pervasive.

The chitosan-canola oil-mineral-water composition can carry these minerals directly to where the body needs them without going through the circulation. This presents a unique delivery system advantage in helping muscles and nerves polarize and depolarize.

The combination of canola oil, chitosan, water and minerals potentiate each ingredient's effect when in combination. They cross the skin barrier and affect all the underlying tissues to stop pain, fight infection and inflammation, help healing and scar formation and boost the immune system. By varying the ion concentration more toward neutral pH by decreasing the amount of ions used can make the invention more soothing on open wounds. By lowering the concentration of minerals, the invention can be used on open wounds for hemostasis and as a matrix for scar formation.

The invention methods of use include, but are not limited to, a solid (canola butter) or an oil (canola oil). Other methods of application include roll-on, application by hand or using cotton swabs, or orally. Other applications include nasal application using cotton swabs or cotton balls to quickly clear up runny nose, sinus congestion and post-nasal drip.

It should be noted that another aspect would be to use the invention for treatment of lacerations, abrasions, spider bites, insect bites, including mosquito bites because when applied topically some of the chitosan which is not transported into the tissue dries into a matrix coat which allows scar tissue to form faster, lessening infection, retarding inflammation and improving immune system function. Applied to mosquito bites it stops the itching and stops the swelling and redness associated with these types of bites.

Another aspect is that the invention can be applied orally to help heal gum infections and aphous ulcers. Oral application is completely safe because none of the components are harmful when ingested orally. Topical application of the composition to the spinal area including neck, midback and lower back over a period of time re-hydrates the intervertebral disc and decreases disc narrowing and tissue shrinkage of old age and disease. Topical application relieves muscle soreness immediately after exercising, eliminates soreness the day after exercising, helps pain of arthritis, heals inflammations of the tendons, ligaments and fascia, lubricates cartilaginous joints such as knees, elbows, hips and shoulders, relieves low back pain and neck stiffness, heals ulcerated tissues caused by spider bites and boosts the immune system.

While the present invention has been described above in terms of specific embodiments, it is to be understood that the invention is not limited to these disclosed embodiments. Many modifications and other embodiments of the invention will come to mind of those skilled in the art to which this invention pertains, and which are intended to be and are covered by both this disclosure and the appended claims It is indeed intended that the scope of the invention should be determined by proper interpretation and construction of the appended claims and their legal equivalents, as understood by those of skill in the art relying upon the disclosure in this specification and the attached drawings.

I claim:

1. A lotion for topical application to skin, comprising a combination of:
canola oil (93 to 94% by weight), chitosan (0.65 to 0.85% by weight), water (4.7 to 5.1% by weight), and sea salt (0.57 to 5.8% by weight).

2. A lotion as in claim 1, wherein:
said sea salt includes, by weight in the lotion, chloride ion (0.34 to 0.54% by weight), sulfate ion (0.009 to 0.0201% by weight), calcium ion (0.0009 to 0.00002% by weight), potassium ion (0.0012 to 0.0032% by weight), zinc ion (0.0000009 to 0.0032% by weight), sodium ion (0.005 to 0.007% by weight), magnesium ion (0.008 to 0.01% by weight), and nickel ion (0.000001 to 0.00002% by weight).

3. A method of making a lotion for topical application to skin, comprising the steps of:
dissolving 7-8 grams of medical or edible grade chitosan into approximately 1 liter of canola oil to create a chitosan-canola oil mixture;
dissolving 2 tsp of sea salt into 50 ml of distilled water to create a sea salt solution;
pouring said sea salt solution into said chitosan-canola oil mixture; and,
vigorously shaking the combined sea salt solution and chitosan-canola mixture for about 10 minutes.

4. A method as in claim 3, wherein:
said sea salt includes in combination the following minerals: chloride ion, sulfate ion, calcium, potassium, zinc, sodium, magnesium and nickel.
